(19) United States
(12) Patent Application Publication
(10) Pub. No.: US 2017/0304107 A1
(43) Pub. Date: Oct. 26, 2017

(54) ORAL APPLIANCE FIXED TO LOWER TEETH

(71) Applicants: Hyun Jin CHOI, Incheon (KR); Pasamedi Co., Ltd., Incheon (KR)

(72) Inventors: Hyun Jin CHOI, Incheon (KR); Young Hyon PARK, Incheon (KR); Jun Young PARK, Incheon (KR); Jun Won PARK, Incheon (KR)

(21) Appl. No.: 15/320,352

(22) PCT Filed: Oct. 24, 2016
(86) PCT No.: PCT/KR2016/011987

§ 371 Date: Jun. 21, 2017

(30) Foreign Application Priority Data
Oct. 22, 2015 (KR) 10-2015-0147532

(51) Int. Cl. A61F 5/56

(52) U.S. Cl. CPC A61F 5/566

(57) ABSTRACT

An oral appliance fixed to lower jaw teeth comprises a body having a shape corresponding to a teeth arrangement, a lower teeth recess formed in a lower portion of the body to allow lower jaw teeth to be inserted therein, an upper teeth recess formed in an upper portion of the body to allow upper jaw teeth to be inserted therein and allowing the lower jaw to move backward by a predetermined distance, and an upper teeth guide part formed to extend from the body to face a side of the upper jaw teeth. The lower teeth recess further comprises a lower molar fixing recess formed to strongly engage with at least a portion of left and right molars of the lower jaw teeth by frictional force, and a lower incisor recess formed to allow other lower jaw teeth except the left and right molars to position therein.

### CROSS-REFERENCE TO RELATED APPLICATIONS

This patent application is a national-stage application of International Patent Application No. PCT/KR2016/011987 filed on Oct. 24, 2016, which claims priority under 35 U.S.C. §119 to Korean Patent Application No. 10-2015-0147542, filed on Oct. 22, 2015, in the Korean Intellectual Property Office, the disclosure of which is incorporated by reference herein in its entirety.

### TECHNICAL FIELD

The present invention relates to an oral appliance, and more particularly, to an oral appliance fixed to lower jaw in which a fixing part to teeth is provided in a lower jaw.

### BACKGROUND ART

Meanwhile, the present inventor has obtained a patent as Korean Patent No. 10-1463021 for an oral appliance fixed to upper jaw teeth.

However, according to the oral state and teeth arrangement of a patient, for example, due to loss of molars of upper jaw teeth, an oral appliance might not be fastened to the upper jaw teeth.

In this case, if a portion to which an oral appliance is fastened is simply changed from upper jaw teeth (molar) to lower jaw teeth (molar), it results in a lot of inconvenience and pain for the patient.

Thus, there is a strong need for an oral appliance which prevents the stiffness of a temporomandibular joint and related muscles and enables a forward movement of the lower jaw teeth by allowing the movement of the lower jaw teeth while fixed to the lower jaw teeth.

### DISCLOSURE OF INVENTION

### Technical Problem

The present invention has been made in response to the demand as above, and an object of the present invention is to provide an oral appliance fixed to lower teeth that may allow the lower jaw smoothly to move even when being worn.

Further, another object of the present invention is to provide an oral appliance fixed to low teeth, which may relax the stiffness of the related muscles and ligaments stiffness caused by a forward movement of the lower jaw by permitting a movement of the low jaw.

Furthermore, still another object of the present invention is to provide an oral appliance fixed to lower teeth that may prevent the occurrence of a pain, a malocclusion or a jaw joint disorder even when worn.

### Solution to Problem

In order to achieve the above-mentioned object, an oral appliance fixed to lower jaw teeth according to an embodiment of the present invention, comprises a body having a shape corresponding to a teeth arrangement, a lower teeth recess formed in a lower portion of the body to allow the lower jaw teeth to be inserted therein, an upper teeth recess formed in an upper portion of the body to allow upper jaw teeth to be inserted therein and allowing the lower jaw to move backward by a predetermined distance, and an upper teeth guide part formed to extend from the body to face a side surface of the upper jaw teeth, wherein the lower teeth recess further comprises a lower molar fixing recess formed to strongly engage with at least a portion of left and right molars of the lower jaw teeth by a frictional force and a lower incisor recess formed to allow other lower jaw teeth except the left and right molars to position therein.

The oral appliance may further comprise a tongue tip mounting part formed on a portion of the body to allow a user's tongue to be placed thereon.

A boundary between the lower molar fixing recess and the lower incisor recess may be a canine.

The upper teeth guide part may be formed such that an extended length of an upper molar region is larger than an extended length of an upper incisor region.

The upper teeth guide part may be formed to extend from the body to a space between a cheek and an upper jaw teeth of a user to face an outer side of the upper jaw teeth.

The upper teeth guide part may be formed to extend toward a palate of a user to face an inner side of the upper jaw teeth.

The upper teeth guide part may comprise a first upper teeth guide part formed to extend from the body to a space between a cheek and an upper jaw teeth of a user to face an outer side of the upper jaw teeth and a second upper teeth guide part formed to extend from the body toward a palate of the user to face an inner side of the upper jaw teeth.

The body may be made of a medical resin or other human-safe material.

The lower incisor recess may be formed to be spaced apart from lower incisors, and thus the lower incisor recess does not contact to the lower incisors.

The upper teeth recess is formed to be spaced apart from upper jaw teeth, and thus the upper teeth recess does not strongly engage with the upper jaw teeth.

The upper teeth recess and the lower teeth recess may be formed such that a lower jaw teeth of a user move forward further than before wearing the oral appliance fixed to the lower jaw teeth.

The lower teeth recess may be formed such that a depth of the lower teeth recess is not larger than a height of an exposed surface of the lower jaw teeth.

### Advantageous Effects of Invention

As described above, according to an embodiment of the present invention, an upper teeth recess or a lower teeth recess in which teeth are inserted provides a gap to be spaced apart from the teeth in the vertical direction and the horizontal direction so that the lower jaw may move smoothly even when an oral appliance is worn.

Further, according to an embodiment of the present invention, the related muscles and ligaments stiffness caused by forward movement of the low jaw may be relaxed by permitting movement of the low jaw.

Furthermore, according to an embodiment of the present invention, a pain, a malocclusion or a jaw joint disorder may not occur even when an oral appliance is worn in contrast to conventional oral appliances.

Moreover, according to an embodiment of the present invention, since air flow through an airway is facilitated, snoring, sleep apnea, rhinitis, or rhinosinusitis may be mitigated.

### BRIEF DESCRIPTION OF DRAWINGS

FIG. 9a is a view illustrating an embodiment regarding an oral appliance fixed to lower jaw teeth,

FIG. 9b is a side view illustrating the oral appliance fixed to lower jaw teeth according to the embodiment illustrated in FIG. 9a,

FIG. 10 is a rear view illustrating a bottom surface of the oral appliance fixed to lower jaw teeth according to the embodiment illustrated in FIG. 9a,

FIG. 11 is a rear view illustrating an upper surface of the oral appliance fixed to lower jaw teeth according to the embodiment illustrated in FIG. 9a,

FIGS. 12 to 14 are cross-sectional views illustrating a molar portion of the oral appliance fixed to lower jaw teeth according to the embodiment illustrated in FIG. 9a,

FIG. 15 is a view from the front right, illustrating an upper surface of the oral appliance fixed to lower jaw teeth according to the embodiment illustrated in FIG. 9a, and

FIG. 16 is a view illustrating the relationship among a tongue, teeth, the oral appliance fixed to lower jaw teeth, and a cross-section of an incisor portion of the oral appliance fixed to lower jaw teeth in a cross-section illustrated in FIG. 15.

### MODE FOR THE INVENTION

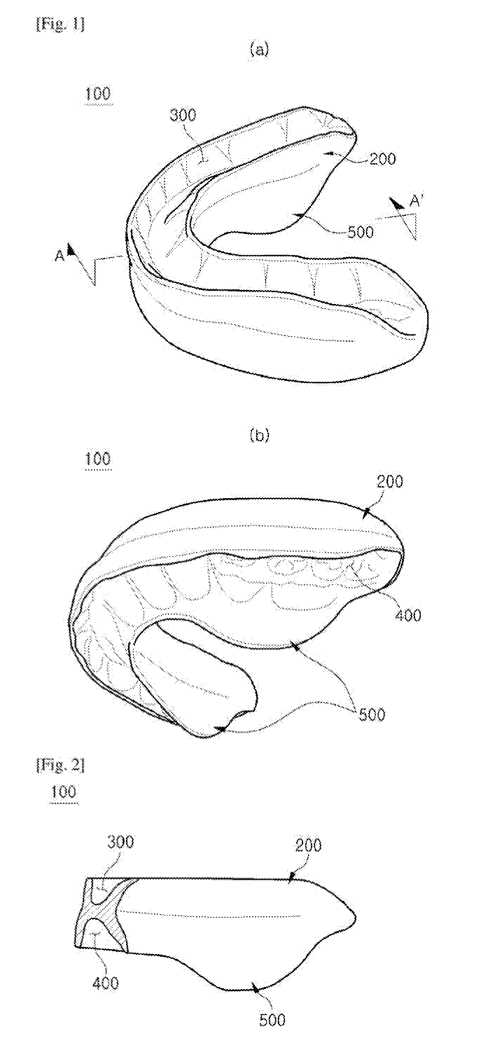

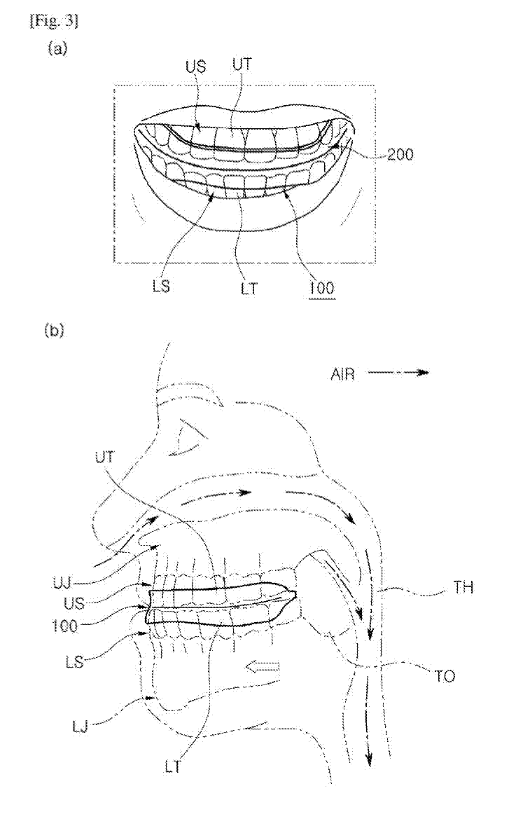

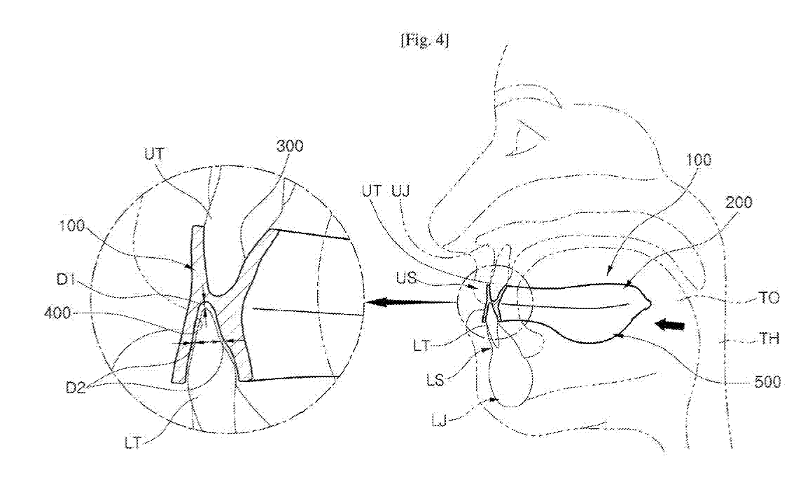

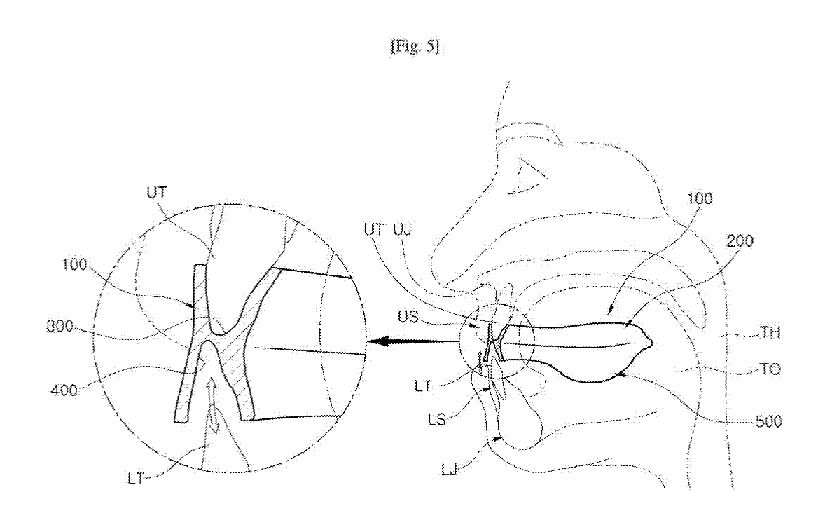

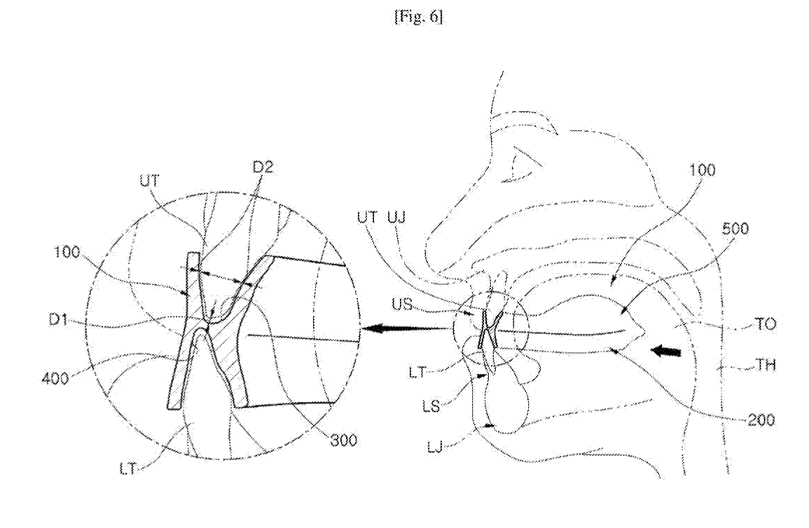

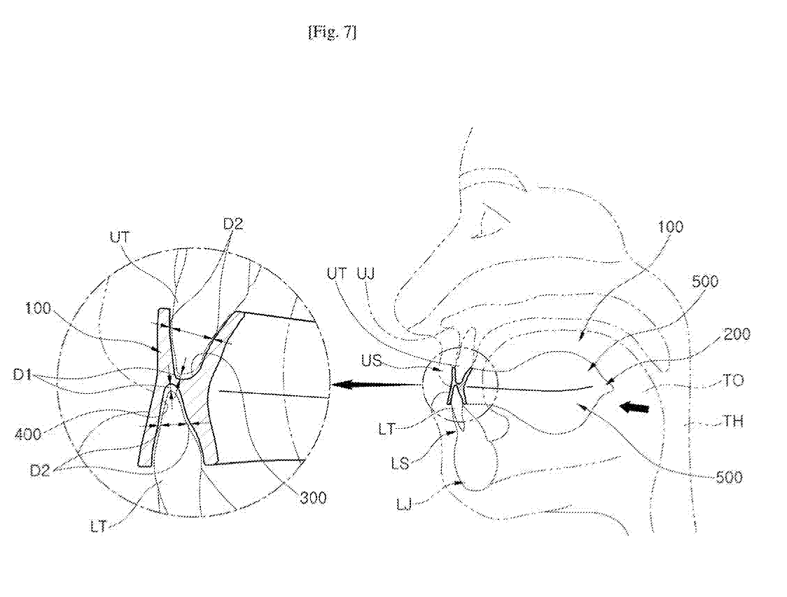

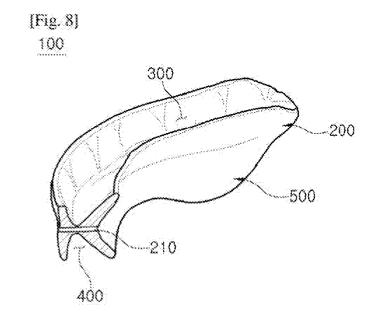

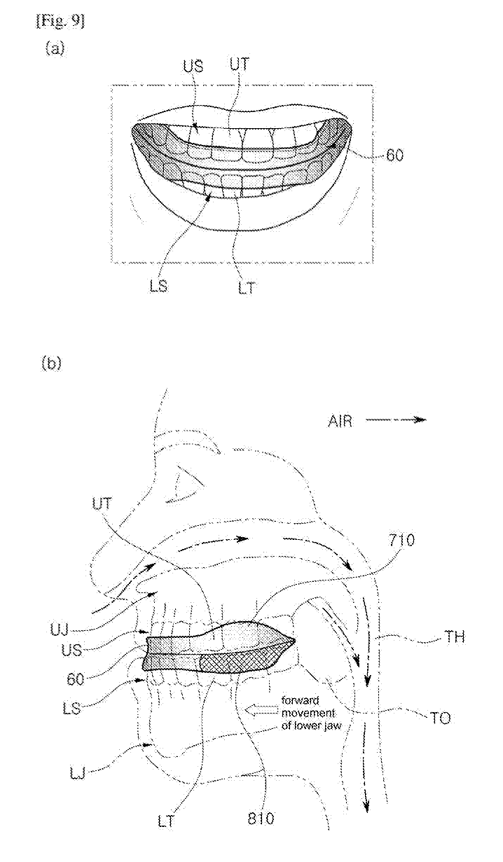

FIG. 9a is a view illustrating an embodiment regarding an oral appliance fixed to lower jaw teeth.

With reference to FIGS. 1 to 8, an oral appliance which is fixed to the upper jaw teeth (molar) has been described above. As illustrated in FIG. 9a, the oral appliance fixed to lower jaw teeth (molar) may also be possible.

As illustrated in FIG. 9a, an oral appliance 60 fixed to lower jaw teeth is similar to an oral appliance 100 fixed to upper jaw teeth when viewed from front of the teeth. However, as described below, the oral appliance fixed to lower jaw teeth 60 has various characteristics different from those of the oral appliance fixed to upper jaw teeth 100.

FIG. 9b is a side view illustrating the oral appliance fixed to lower jaw teeth according to the embodiment illustrated in FIG. 9a.

As illustrated in FIG. 9b, one most noticeable feature of the oral appliance fixed to lower jaw teeth 60 is that the oral appliance 60 is fixed to a portion of the lower jaw teeth LT of a user. Particularly, at least one lower jaw tooth LT in the left and right molar regions among the lower jaw teeth LT of a user is fixed to a lower molar fixing recess 810 by a frictional force.

Meanwhile, the upper jaw teeth arrangement US is not fixed to the oral appliance fixed to lower jaw teeth 60. Accordingly, the upper jaw teeth arrangement US may freely move within a predetermined range. Further, an upper teeth guide part 710 is formed so that even if the upper jaw teeth arrangement US moves, the upper jaw teeth arrangement US may be positioned in place inside an upper teeth recess 700 of a body 600. The upper teeth guide part 710 is formed to contact a side surface of the upper law teeth.

FIG. 10 is a rear view illustrating a bottom surface of the oral appliance fixed to lower jaw teeth according to the embodiment illustrated in FIG. 9a, and FIG. 11 is a rear view illustrating an upper surface of the oral appliance fixed to lower jaw teeth according to the embodiment illustrated in FIG. 9a.

Each part constituting the oral appliance 60 fixed to lower jaw teeth will be described in more detail with reference to FIGS. 10 and 11.

The oral appliance 60 fixed to lower jaw teeth is configured by forming a body 600 with a material, such as a medical resin and other material that is safe for human body. The body 600 further comprises an upper teeth recess 700, an upper teeth guide part 710, a lower teeth recess 800, and a tongue tip mounting part 900.

Meanwhile, the lower teeth recess 800 further comprises lower molar fixing recesses 811 and 812 in which a molar portion of lower teeth is positioned and a lower incisor recess 820 in which an incisor portion of lower teeth is positioned.

At least one of the left and right molars of lower jaw teeth is strongly engaged with the lower molar fixing recesses 811 and 812 by a frictional force. On the other hand, the incisor portion of the lower jaw teeth is positioned in the lower incisor recess 820, but the incisor portion is not engaged with an inner side of the lower incisor recess 820.

A boundary between the lower molar fixing recess 811 and the lower incisor recess 820 is a left canine, and a boundary between the lower molar fixing recess 812 and the lower incisor recess 820 is a right canine.

Meanwhile, the upper jaw teeth UT and the lower jaw teeth LT respectively are positioned in the upper teeth recess 700 and the lower teeth recess 800. Here, the lower teeth recess 800 is formed to be positioned ahead of the normal occlusal surface of a user so that the lower jaw teeth LT of the user wearing the oral appliance 60 move forward further than before wearing the oral appliance. As the user's lower jaw teeth LT move forward, the cross-sectional area of the user's airway widens, improving the air flow during respiration. This helps prevent or alleviate snoring while the user sleeps. It also helps to relieve the user of inflammation, such as rhinitis and sinusitis.

Upper teeth guide parts 711 and 712 are formed so that the upper jaw teeth arrangement US may be located in place in the upper teeth recess 700 of the body 600 even if the upper jaw teeth arrangement US moves.

As described above, the upper jaw teeth arrangement US is strongly engaged with the upper teeth recess 700 or is not fixed to the inside of the upper teeth recess 700. Accordingly, the upper jaw teeth arrangement US may freely move within a predetermined range departing from the upper teeth recess 700. Thus, although the oral appliance 60 is worn for a long time, muscles and ligaments around the oral cavity may not be rigid. Further, pain, malocclusion, or temporomandibular joint disorders caused by the stiffness of muscles and ligaments around oral cavity may not occur.

However, since the upper jaw teeth arrangement US should be able to locate in the upper teeth recess 700, the upper teeth guide parts 711 and 712 are formed to extend from the body 600 at a portion where the upper teeth guide parts 711 and 712 contact a side surface of an upper jaw teeth UT. Further, the upper teeth guide parts 711 and 712 may be formed such that an extended length of an upper molar region is larger than an extended length of an upper incisor region.

Further, the tongue tip mounting part 900 is formed as a cavity so that a tongue tip of a user may stably be seated ahead of the oral appliance 60.

Various conventional oral devices, such as an oral appliance for preventing snoring, do not provide a space for accommodating the user's tongue. The user is unable to stably rest the tongue, and thus the tongue moves back to the original rear position, and the adaptability to the oral appliance is lowered.

In order to solve such a problem, an oral appliance fixed to lower jaw teeth 60 comprises the tongue tip mounting part 900 allowing the user's tongue tip to be stably placed ahead.

FIGS. 12 to 14 show three types of cross-sectional views illustrating a molar portion of the oral appliance fixed to lower jaw teeth according to the embodiment illustrated in FIG. 9a,

Each of FIGS. 12 to 14 is a cross-sectional view illustrating a plane in the normal direction of a front side of a user, which is one of planes including the straight line connecting a left-side molar portion (A′ point) and right-side molar portion (A point) of the user in FIG. 11.

As illustrated in FIG. 12, the lower jaw left-side molar and the lower jaw right-side molar, respectively, are positioned in inner sides of lower molar fixing recesses 811 and 812. Further, at least one lower jaw molar is strongly engaged with the inner side of the lower molar fixing recesses 811 and 812 to prevent the oral appliance 60 from too easily escaping off from a user.

As described above, upper jaw molars are positioned in an upper teeth recess 700, but are not strongly engaged with an inner side of the upper teeth recess 700. Further, the upper teeth guide parts 711 and 712, respectively, are formed in the right and left of the upper teeth recess 700 of the body 600 so that upper jaw molars may escape off from the upper teeth recess 700 and then come back to their position in the upper teeth recess 700.

Further, as illustrated in FIG. 13, upper teeth guide parts 713 and 714, respectively, are formed in the right and left of the upper teeth recess 700 of the body 600 so that upper jaw molars may escape off from the upper teeth recess 700 and then come back to their sites in the upper teeth recess 700.

In other words, the upper teeth guide parts 713 and 714 are formed to extend from the body 600 toward the palate of the user to face an inner side of upper jaw teeth.

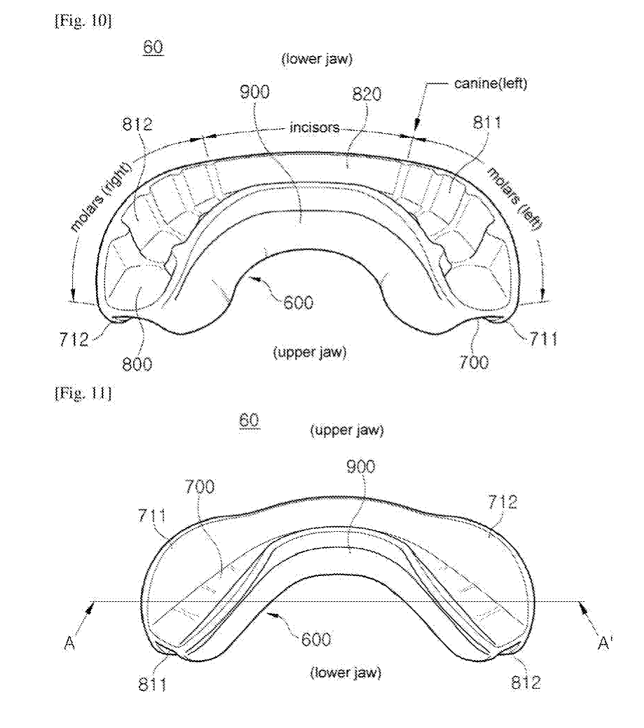

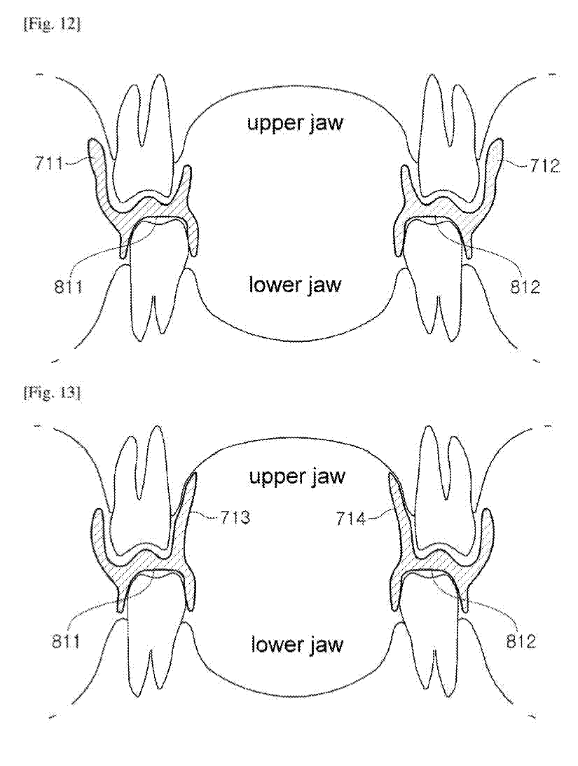

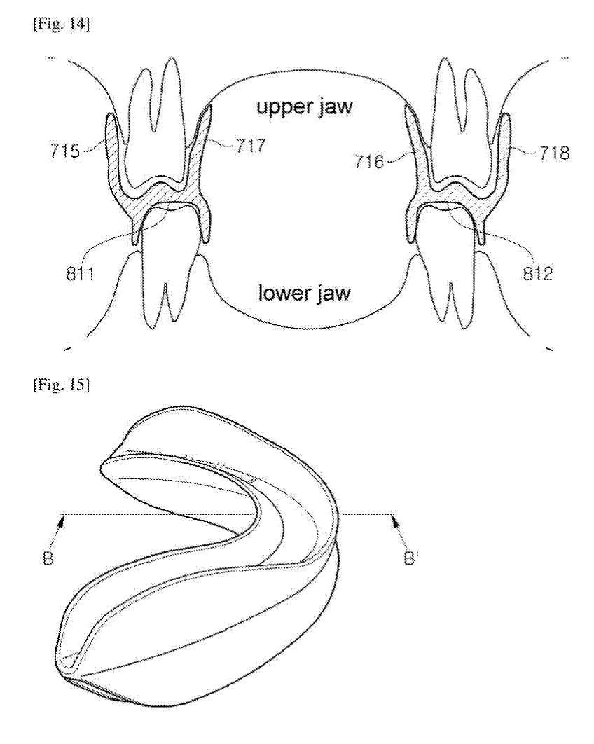

Further, as illustrated in FIG. 14, upper teeth guide parts 715, 716, 717 and 718, respectively, may be formed in the right and left of the upper teeth recess 700 of the body 600 so that upper jaw molars may escape off from the upper teeth recess 700 and then come back to their position in the upper teeth recess 700.

In other words, first upper teeth guide parts 715 and 718 disposed to face an outer side surface of upper jaw teeth are formed to extend from a body 600 to a space between a cheek and an upper jaw teeth of a user. Further, second upper teeth guide parts 716 and 717 disposed to face an inner side of upper jaw teeth are formed to extend from the body 600 toward a palate of a user.

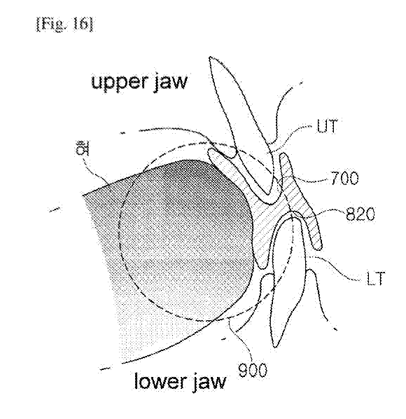

FIG. 15 is a view from the front right, illustrating an upper surface of the oral appliance fixed to lower jaw teeth according to the embodiment illustrated in FIG. 9a, and FIG. 16 is a view illustrating the relationship among a tongue, teeth, the oral appliance fixed to lower jaw teeth, and a cross-section of an anterior teeth part of the oral appliance fixed to lower jaw teeth in a cross-section illustrated in FIG. 15.

The cross-sectional view of FIG. 16 is a cross-sectional view illustrating a plane including the straight line connecting front and rear portions of a user among planes including the straight line connecting an upper incisor portion (B point) and a lower incisor portion (B′ point) shown in FIG. 15.

As illustrated in FIG. 16, the upper jaw incisor UT is positioned in the upper teeth recess 700, and the lower jaw incisor LT is positioned in the lower incisor recess 820.

Neither the upper teeth recess 700 nor the lower incisor recess 820 engages strongly with teeth. Further, as described above referring to FIGS. 10 and 11, the upper jaw incisor UT and the lower jaw incisor LT respectively are positioned in the upper teeth recess 700 and the lower incisor recess 820. Here, the lower teeth recess 800 is formed to be positioned ahead of the normal occlusal surface of a user so that the lower jaw teeth LT of the user wearing the oral appliance 60 move forward further than before wearing the oral appliance. As the user's lower jaw teeth LT moves forward, the cross-sectional area of the user's airway is widened, so that the air flow during respiration is improved.

Further, a user's tongue is placed on the tongue tip mounting part 900 formed on the body 600. It is preferable that the surface of the tongue tip mounting part 900 is smoothed so as not to cause inconvenience or discomfort to a user. Furthermore, it is preferable that the tongue tip mounting part 900 is formed to be curved along with curve of the tongue tip.

As described above, according to the present invention, although when wearing an oral appliance fixed to lower jaw teeth, it is possible to implement an oral appliance that allows lower jaw teeth naturally to move.

Further, it is possible to implement an oral appliance that allows the related muscles and ligaments stiffness caused by forward advancement of the low jaw to be relaxed by permitting movement of the low jaw.

Furthermore, it is possible to implement an oral appliance that enables a pain, a malocclusion or a jaw joint disorder not to occur even when an oral appliance is worn.

Accordingly, since air flow through an airway of a user is more facilitated, symptoms of snoring, sleep apnea, rhinitis, or rhinosinusitis may be improved.

The oral appliance described above may be applied not only to the configuration of the above-described embodiments but also to all or part of the embodiments so that various modifications of the embodiments may be made.

What is claimed is:

1. An oral appliance fixed to lower jaw teeth comprising:
a body having a shape corresponding to a teeth arrangement;
a lower teeth recess formed in a lower portion of the body to allow the lower jaw teeth to be inserted therein;
an upper teeth recess formed in an upper portion of the body to allow upper jaw teeth to be inserted therein and allowing the lower jaw to move backward by a predetermined distance; and
an upper teeth guide part formed to extend from the body to face a side surface of the upper jaw teeth, wherein the lower teeth recess further comprises a lower molar fixing recess formed to strongly engage with at least a portion of left and right molars of the lower jaw teeth by a frictional force and a lower incisor recess formed to allow other lower jaw teeth except the left and right molars to position therein.

2. The oral appliance of claim 1, further comprising a tongue tip mounting part formed on a portion of the body to allow a user's tongue to be placed thereon.

3. The oral appliance of claim 1, wherein a boundary between the lower molar fixing recess and the lower incisor recess is a canine.

4. The oral appliance of claim 1, wherein the upper teeth guide part is formed such that an extended length of an upper molar region is larger than an extended length of an upper incisor region.

5. The oral appliance of claim 4, wherein the upper teeth guide part is formed to extend from the body to a space between a cheek and an upper jaw teeth of a user to face an outer side of the upper jaw teeth.

6. The oral appliance of claim 4, wherein the upper teeth guide part is formed to extend toward a palate of a user to face an inner side of the upper jaw teeth.

7. The oral appliance of claim 4, wherein the upper teeth guide part comprises:
a first upper teeth guide part formed to extend from the body to a space between a cheek and an upper jaw teeth of a user to face an outer side of the upper jaw teeth; and
a second upper teeth guide part formed to extend from the body toward a palate of the user to face an inner side of the upper jaw teeth.

8. The oral appliance of claim 1, wherein the body is made of a medical resin or other human-safe material.

9. The oral appliance of claim 1, wherein the lower incisor recess is formed to be spaced apart from lower incisors, and thus the lower incisor recess does not contact to the lower incisors.

10. The oral appliance of claim 1, wherein the upper teeth recess is formed to be spaced apart from upper jaw teeth, and thus the upper teeth recess does not strongly engage with the upper jaw teeth.

11. The oral appliance of claim 1, wherein the upper teeth recess and the lower teeth recess are formed such that a lower jaw teeth of a user move forward further than before wearing the oral appliance fixed to the lower jaw teeth.

12. The oral appliance of claim 1, wherein the lower teeth recess is formed such that a depth of the lower teeth recess is not larger than a height of an exposed surface of the lower jaw teeth.
